(19) United States
(12) Patent Application Publication
(10) Pub. No.: US 2026/0363893 A1
(43) Pub. Date: Nov. 19, 2026

(54) ORTHOPEDIC WALKER AND METHOD OF MANUFACTURING

(71) Applicant: Ovation Systems d/b/a Ovation Medical, Inc., Agoura Hills, CA (US)

(72) Inventor: Kenji Watabe, Ventura, CA (US)

(73) Assignee: Ovation Systems d/b/a Ovation Medical, Inc., Agoura Hills, CA (US)

(21) Appl. No.: 19/668,091

(22) Filed: May 5, 2026

Related U.S. Application Data
(60) Provisional application No. 63/805,697, filed on May 14, 2025.

(51) Int. Cl. A61F 5/058

(52) U.S. Cl. CPC A61F 5/0585

(57) ABSTRACT

The present invention is an orthopedic walker and method of manufacture wherein the walker includes a plastic base and a foam sole that are integrated together during the manufacturing process. The foam sole is formed of a polyurethane foam that bonds both mechanically and/or chemically to the plastic base. The base of the walker at the lower surface is formed with a series of slats defining openings therebetween. During the manufacturing process, polyurethane foam is introduced at the base’s lower surface and flows over the lower surface and through the openings defined by the slats. When the foam dries and hardens, the slats are captured by the foam, which forms a strong mechanical bond which also can benefit from a chemical bond with the slats and the lower surface of the base.

### CROSS-REFERENCES TO RELATED APPLICATIONS

This application claims priority to U.S. Provisional Patent Application No. 63/805,697, filed May 14, 2025, the content of which is incorporated by reference herein in its entirety.

### BACKGROUND

Orthopedic walking boots, also known as orthopedic boots or “walkers,” are specialized footwear designed to provide support, stability, and protection to the foot and ankle during recovery from injury or surgery. These boots are commonly used for conditions such as fractures, sprains, ligament injuries, or after surgical procedures to ensure proper healing. They are typically made with a rigid or semi-rigid shell, adjustable straps, and a cushioned interior to immobilize the affected area, preventing further injury and reducing pain during movement.

The primary benefit of orthopedic walking boots is their ability to stabilize the foot and ankle, which promotes proper healing and minimizes the risk of complications. By immobilizing the injured area, these boots help reduce inflammation, prevent excessive movement that could worsen the injury, and allow the tissues to recover more effectively. Additionally, they help distribute weight evenly, alleviating pressure on vulnerable parts of the foot or ankle, which can significantly improve comfort and mobility during the recovery process. This stabilization often enables patients to bear weight on the affected limb sooner than they could with traditional bandages or less supportive footwear.

Beyond physical support, orthopedic walking boots also offer psychological benefits by allowing patients to maintain a more active lifestyle during recovery. Instead of being confined to bed or crutches, patients can often walk with the assistance of the boot, which can improve mental well-being and reduce feelings of dependency. Moreover, these boots are typically designed to be adjustable and removable, making hygiene and skin care easier, thereby decreasing the risk of skin irritation or infections. Overall, orthopedic walking boots are a crucial tool in modern injury management, facilitating faster recovery while enhancing comfort and mobility for patients.

Although orthopedic walking boots have been on the market for years, their construction and manufacturing process can be problematic. A pad or “sole” is usually glued onto the lower surface to create traction for the patient, but extended use can cause the pad to separate from the plastic molding and lead to premature failure. The present invention provides a solution that results in bond between the sole and the boot to prevent separation while forming a lightweight and robust walker.

### SUMMARY OF THE INVENTION

The present invention is an orthopedic walker and method of manufacture wherein the walker includes a plastic base and a foam sole that are integrated together during the manufacturing process. The foam sole is formed of a polyurethane foam that bonds both chemically and/or mechanically to the plastic base. The base of the walker at the lower surface is formed with a series of slats defining openings therebetween. During the manufacturing process, polyurethane foam is introduced at the base’s lower surface and flows over the lower surface and through the openings defined by the slats. When the foam dries and hardens, the slats are captured by the foam, which forms a mechanical bond with the slats and the lower surface of the base. The foam also replaces the existing shock absorbing insert or foot pad inside the walker. The coupling of the sole and base through chemical bonding and/ or mechanical bonding (slats captured by the hardened foam) results in a walker that withstands greater shear and compressive forces without separation from the base, resulting in a stronger and more reliable walker.

In the manufacturing of the walker of the present invention, the base is placed into the mold and foam is introduced around and through the bottom surface, interfacing with and surrounding the slats formed in the lower surface. A rubber layer can be added for traction and durability purposes prior to the foam sole is formed. The rubber layer can be poured into the mold in a form or separating dam or without. The foam can also be poured over the rubber layer to encapsulate the entire structure into a single, solid unit.

These and other benefits of the present invention will best be understood from the detailed description of the inventors’ best modes of making and using the invention set forth below, in conjunction with the accompanying drawings.

### BRIEF DESCRIPTION OF THE DRAWINGS

FIG. 1 is an elevated, perspective view of a base of the present invention;

FIG. 2 is a partially exploded side view of the present invention;

FIG. 3 is a side view of the present invention;

FIG. 4 is an elevated perspective view of a mold used to create the base of FIG. 1;

FIG. 5 is an elevated perspective view of the base and the mold; and

FIG. 6 is a bottom view of the present invention.

### DETAILED DESCRIPTION OF THE PREFERRED EMBODIMENTS

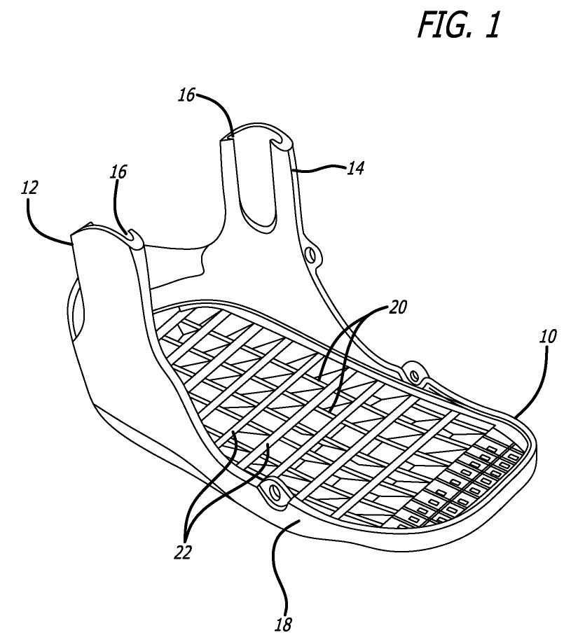

The present invention is an orthopedic walker 100 having a novel base 10 that provides increased stability and improves product durability. The base is formed of a substantially rigid polymer that provides some flexibility while satisfying the supporting function of the device. FIG. 1 illustrates the base 10, which comprises left and right extensions 12, 14 with inner slots 16 configured to mount vertical struts (not shown). The base 10 includes a foot bed 18 sized to receive a patient’s foot thereon, where the foot bed 18 is formed by parallel vertical longitudinal slats 20 and parallel, transverse, forwardly angled slats 22. The resulting structure is a series of repeating oblique parallelepiped channels defining an array that extend through the base’s lower surface and provide pathways from the upper to lower surfaces of the foot bed. In a preferred embodiment the parallel, transverse forwardly angled slats form an angle of approximately forty-five degrees with a horizontal surface such as the ground. A perimeter of the footbed includes an upper edge that rises slightly above the array of slats 20,22.

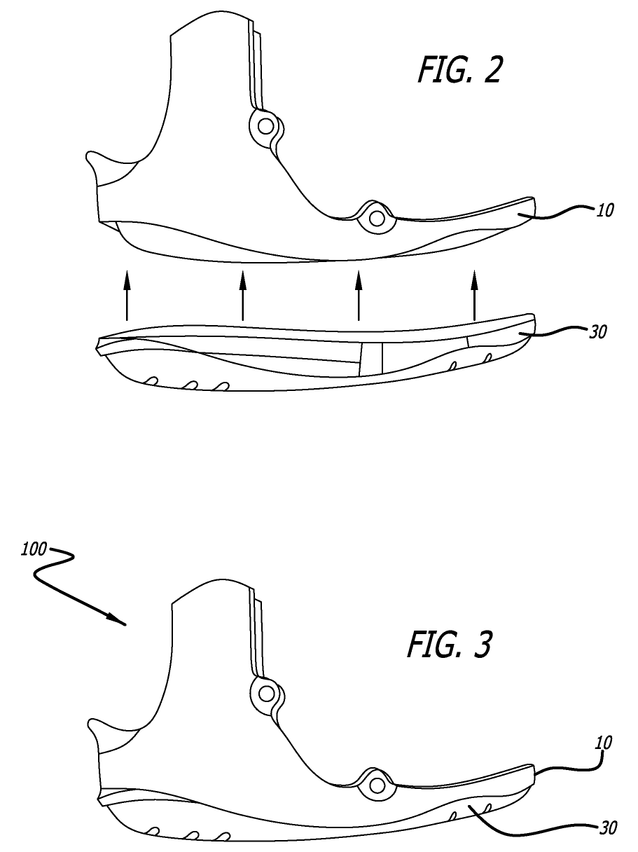

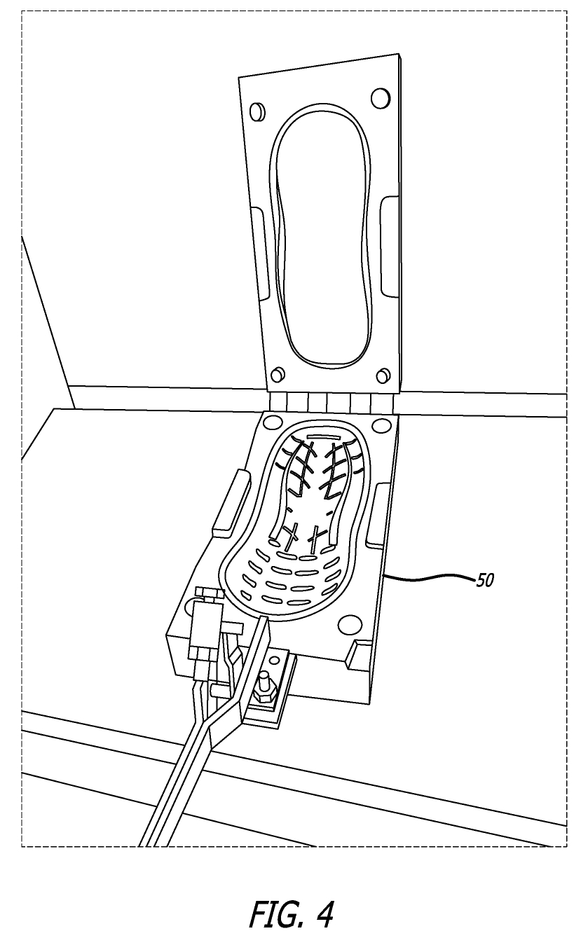

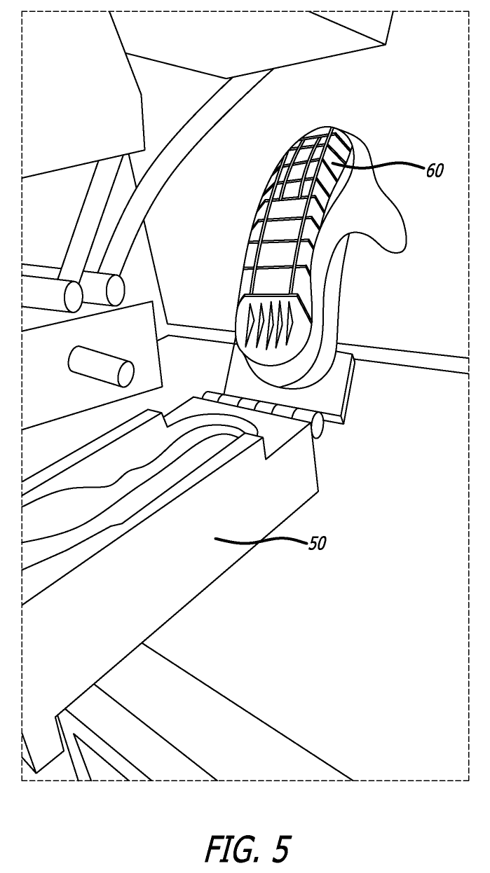

In the manufacturing process, the base 10 is placed in a mold 50 (FIG. 4) in order to apply and adhere a foam sole 30 depicted in FIGS. 2 and 3. The foam is poured into the mold 50, and then the base 10 is pressed inside, allowing the foam to surround the lower surface and flow between/through the slats to form a overlayer that sandwiches the slats above and below. The foam is selected to mechanically and/or chemically bond to the plastic base as the foam transcends the slats to form a foam inner layer inside the walker as well as the outer cushioning layer that absorbs shock when the use walks and the walker strikes the ground.

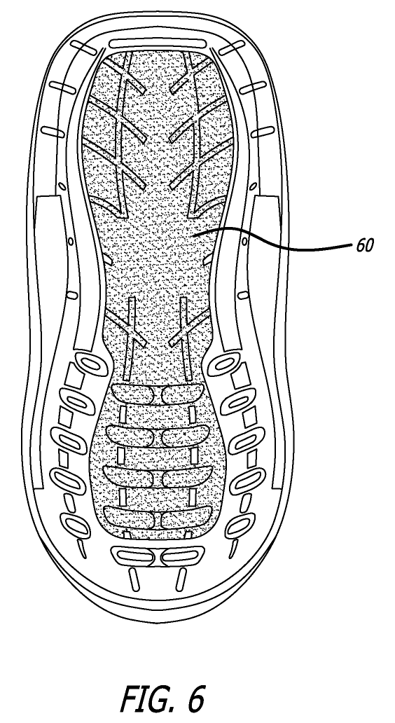

The 50 includes slots to accommodate the left and right extensions so that the base 10 is seated into the upper portion of the mold. Prior to the introduction of the base 10, the mold 50 is cleaned and sprayed with compressed hot air, which raises the temperature of the mold to between 50 - 55°C. A lubricant is sprayed into the mold to prevent the foam from sticking to the mold itself and then the base is placed in the mold. Here, the mold can have different pathways for different materials that lead to different areas of the base via channels and dams. The base, located in the upper portion of the mold, is lowered into the mold where it is introduced into the lower mold interior and where the foam has been poured, and then the mold is closed. Heat is applied until the mold reaches a temperature of 100° C for up to four minutes. The mold is then allowed to cool, and base/sole walker combination is removed and trimmed as part of the finishing process. A molten rubber layer 60 can be applied to the mold, especially in areas where greater traction is needed (FIG. 6). This can be poured directly into the lower portion of the mold or within a dammed area to maintain shape.

The foam is preferably a polyurethane specially formulated for high rebound, enhanced bonding to plastic, durability, and surface grip. As the material makes its way through the openings formed by the slats 20.22, the material mechanically bonds to the plastic features and can also chemically bonds to the surfaces of the base 10. This is a departure from traditional adhesive techniques to join the two elements.

The benefit of the foregoing device and method of manufacture is an orthopedic walker that is better suited for the wear and tear a walker faces when used on hard or abrasive surfaces. The walker is lightweight, sturdy, and can be molded together in a single process that eliminates the need for fasteners, inserts, and foam padding. The process eliminates 9 pcs from the walker current assembly including a separate shock absorber, insole plate, six fasteners and insole EVA foam layer.

While the preferred embodiment of the invention has been depicted and described above, the invention is not limited to the foregoing descriptions and depictions. A person of ordinary skill in the art will readily recognize various modifications and substitutions, and the present invention is intended to include all such modifications and substitutions. Nothing in this description should be deemed as limiting or exclusionary unless expressly so stated.

1. An orthopedic walker, comprising:
a base having a lattice of intersecting slats defining open channels therebetween, said intersecting slats comprising vertical longitudinal slats and forward slanted transverse slats;
a foam footbed injected about the base such that the open channels are filled with said foam and sandwiches the base completely on an upper and lower surface, said foam footbed having recesses imbedded therein on a bottom portion;
vertical supports extending from the base on left and right sides thereof; and
a polymer lowermost layer molded into the foam footbed’s recesses to provide a durable ground contact surface.

2. The orthopedic walker of claim 1, wherein the channels are angled at an approximate inclination of forty-five degrees.

3. The orthopedic walker of claim 1, wherein the foam is selected to chemically bond with the base.

4. The orthopedic walker of claim 3, wherein the foam is a polyurethane.
